(19) United States
(12) Patent Application Publication
(10) Pub. No.: US 2010/0210018 A1
(43) Pub. Date: Aug. 19, 2010

(54) MICROBIAL HOST-VECTOR COMPLEMENTATION SYSTEM

(75) Inventors: Gary Gates, Hatboro, PA (US); Chandrasekhar Satishchandran, Worcester, MA (US)

Correspondence Address: RATNERPRESTIA, P.O. BOX 980, VALLEY FORGE, PA, 19482, US

(73) Assignee: Veritas Bio LLC, Plymouth Meeting, PA (US)

(21) Appl. No.: 12/596,793

(22) PCT Filed: Apr. 21, 2008
(86) PCT No.: PCT/US08/61024

§ 371 Date: Apr. 16, 2010

Related U.S. Application Data
(60) Provisional application No. 60/907,901, filed on Apr. 20, 2007.

(51) Int. Cl. C12N 15/74; C12N 15/63; C12N 1/00

(52) U.S. Cl. 435/476; 435/471; 435/320.1; 435/243

(57) ABSTRACT

This invention provides a host-vector complementation system, which permits selection of vector-carrying host cells, without requiring an antibiotic resistance gene. In some embodiments, this system utilizes a host which is guaB deficient and vectors that carry and express the guaB gene. The invention also discloses methods of making and using the system.

### CROSS REFERENCE TO RELATED APPLICATIONS

This application claims priority to U.S. Provisional Application No. 60/907,901, filed Apr. 20, 2007, which is hereby incorporated by reference in its entirety.

### FIELD OF THE INVENTION

The present invention relates to a novel microbial/vector selection system that does not require or involve an antibiotic resistance gene.

### BACKGROUND

Nucleic acids, including DNA plasmid expression vectors, constitute an exciting modality for pharmaceutical applications. Nucleic acid expression vectors can be engineered to transcribe biologically active nucleic acids such as eiRNA or RNAi molecules (expressed double stranded RNA (dsRNA)), expressed antisense, as well as various biologically active polypeptide and protein molecules useful for gene therapy, DNA vaccines, etc.

Nucleic acid expression constructs or expression vectors, e.g., plasmid expression vectors, are frequently produced by fermentation in a bacterial or other unicellular host (cell culture), particularly E. coli. Plasmids to be grown in a bacterial or other unicellular host often are engineered to carry a selection marker, such as an antibiotic resistance gene, to allow selection for host cells containing the plasmid during manufacturing. However, for human or veterinary applications, a plasmid capable of conferring antibiotic resistance to microorganisms would be undesirable.

### SUMMARY OF THE INVENTION

The present invention is a host-vector complementation system, which permits selection of vector-carrying host cells, without relying on an antibiotic resistance gene.

GuaB encodes the gene product IMPDH, or inosine monophosphate dehydrogenase. IMPDH is an enzyme critical for synthesis of guanosine triphosphate (GTP), a molecule required for DNA synthesis and cellular signaling in host cells. IMPDH catalyzes the first step in the de novo synthetic pathway for the formation of guanine nucleotides by converting IMP to xanthosine 5′-monophosphate (XMP) with the concomitant reduction of NAD⁺. This is the rate-limiting step in this pathway and the enzyme is therefore an important regulator of cell proliferation. A guaB⁻ microorganism will not grow in the absence of guanine, such as, for example, on minimal medium.

In one aspect, the present invention provides a method for selecting recombinant microorganisms. The method comprises transforming a host microorganism having an inactive, substantially inactive, or deleted guaB gene(s) (for example, via one or more mutations in guaB), with a vector. The vector is generally a recombinant vector, such as an expression vector, and may contain one or more polynucleotide sequence(s) of interest. For example, the vector may encode and direct the expression of an RNA molecule or polypeptide useful for therapeutic or research applications. In accordance with the invention, the vector complements the host microorganism's guaB deficiency, generally by providing a functional guaB gene. Transformed microorganisms may be grown on media deficient in guanine, such as minimal media, so as to permit the selective growth of microorganisms carrying the complementing vector. In certain embodiments, the vector is a plasmid, such as an autonomously replicating plasmid, or is a viral vector. In accordance with this aspect of the invention, the presence of an antibiotic selection marker is rendered unnecessary.

In a second aspect, the invention provides a vector comprising a functional guaB gene. The vector may further comprise at least one polynucleotide sequence of interest, for example as described herein, or may contain convenient restriction sites for insertion of such as sequence to be operably controlled by a promoter. Generally, the vector does not contain a functional antibiotic resistance gene, or does not encode or express gene products necessary for imparting antibiotic resistance to a host microorganism. The vector is sufficient for complementing a host microorganism's guaB deficiency or inactivation (e.g., as described herein). In various embodiments, the vector may be a plasmid or a viral vector, and may be suitable for replication in one or more bacterial or eukaryotic hosts, including Escherichia coli, Bacillus subtilis, and Salmonella; and yeasts such as Saccharomyces cerevisiae and Pichia pastoris. In accordance with this aspect, the presence of an antibiotic selection marker on the vector is unnecessary, making the vector desirable for pharmaceutical applications particularly. Thus, in a related aspect, the present invention provides a pharmaceutical composition comprising a vector of the invention, and a pharmaceutically acceptable delivery vehicle.

In a further aspect, the present invention provides for an expression system comprising a host microorganism containing an inactive or substantially inactive guaB gene, and a vector of the invention carrying a recombinant gene, or suitable for carrying a recombinant gene. Generally, the vector is suitable for complementing the host's inactivation or substantial inactivation of guaB. The vector may be contained within the host microorganism, so as to produce an RNA or protein of interest within the microorganism. Such microorganism find use as pharmaceutical compositions, and in their production, obviate the need for selection based upon antibiotic resistance. Alternatively, the vector and microorganism may be provided separately, for example, as a kit, for various research and pharmaceutical applications.

### BRIEF DESCRIPTION OF THE FIGURES

FIG. 1. Exemplary cloning strategy for replacement of kanamycin resistance gene (kanR gene) with guaB gene in shRNA-expressing plasmid.

FIG. 2. Exemplary E. coli guaB gene sequence with 5′ and 3′ flanking regions (SEQ ID NO: 1), and encoded amino acid sequence (SEQ ID NO: 2).

FIG. 3. The chromosomal situation of guaB in the E. coli genome. Homology arms are boxed. Exemplary check primers for subsequent PCR verification of correct insertion events are underlined (SEQ ID NO: 3).

FIG. 4. Results of a check PCR performed using primers located on the chromosome and primers located in the cassette, and showing the expected results. Lanes are as follows: (1) size standards; (2) check_up2/check_down1 recA (226 bps); (3) check_up1/check_down1 endA (229 bps); (4) check1/GBR14 guaB (393 bps); (5) check_up2/check_down1 recA (226 bps); (6) check_up1/check_down1 endA (229 bps); (7) check_up1/check_down1 sbsCD (226 bps); (8) check 1/GBR14 guaB (393 bps).

FIG. 5. Results of a guaB chromosomal deletion, after deletion and removal of the selection marker, and showing the expected results. Lanes are as follows: (1) size standards; (2) guaB check1/check3 (236 bps) triple mutant; (3) guaB check1/check3 (236 bps) quadruple mutant.

### DETAILED DESCRIPTION

The inventors have engineered clinically relevant vectors that do not require or include a functional antibiotic resistance gene, and which can be replicated in a suitable host cell, including a bacterial host cell such as E. coli, without an antibiotic resistance selection. The vectors contain an essential endogenous gene of the host cell involved in production of GTP, such as guaB. Thus, the invention provides for expression systems involving a host microorganism containing an inactivated, substantially inactivated, or deleted guaB gene (e.g., a guaB⁻ host), and a vector that contains a functional guaB gene without an antibiotic resistance marker.

The guaB gene is relatively small in size (about 700 bp), and thus may be accommodated by various expression vectors, including plasmid and viral vectors. guaB encodes the gene product IMPDH, inosine monophosphate dehydrogenase. IMPDH is an enzyme critical for synthesis of guanosine triphosphate (GTP), a molecule required for DNA synthesis and cellular signaling. IMPDH catalyzes the first step in the de novo synthetic pathway for the formation of guanine nucleotides by converting IMP to xanthosine 5′-monophosphate (XMP) with the concomitant reduction of NAD⁺. This is the rate-limiting step in this pathway and the enzyme is therefore an important regulator of cell proliferation. A guar microorganism, which does not carry the guaB plasmid or which carries an inactive or substantially inactive guaB gene, does not grow well on medium deficient in guanine, such as minimal medium.

### Selection Methods

In one aspect, the invention comprises a method for selecting recombinant microorganisms. The method comprises transforming a host microorganism having an inactive, substantially inactive, or deleted guaB gene(s) (for example via one or more mutational events in guaB), with a vector. The vector is generally a recombinant vector, such as an expression vector, and may contain one or more polynucleotide sequence(s) of interest. For example, the vector may encode and direct the expression of one or more RNA molecule(s) and/or polypeptide(s) useful for therapeutic or research applications (e.g., siRNA, shRNA, protein biologic, antigen, etc.). In accordance with the invention, the vector complements the host microorganism's guaB deficiency by providing a functional guaB gene. Transformed microorganisms may be grown on media deficient in guanine, such as minimal media, so as to permit the selective growth of microorganisms carrying the complementing vector. In certain embodiments, the vector is a plasmid, such as an autonomously replicating plasmid, or is a viral vector. In accordance with this aspect of the invention, the presence of an antibiotic selection marker is rendered unnecessary.

The host cells, once contacted (e.g., via electroporation or other transformation or transfection technique) with the vector harboring a functional guaB gene, are grown under conditions that provide selective pressure for vector-containing cells. Such conditions may include growth on media deficient in guanine, such as minimal media, thereby encouraging production and retention of the vector during fermentation (growth). Together the vector and host constitute a fermentation system for production of plasmids or other expression vectors that, desirably, do not contain an antibiotic resistance gene or a functional antibiotic resistant gene. In certain embodiments, the host microorganism may contain a guaB mutation, such as a knockout of all, or a functional portion of, guaB. In another embodiment, the guaB mutation is a deletion or insertion of a polynucleotide sequence at the guaB locus sufficient to inactivate the gene, or any other genetic variation that results in substantially decreased expression (e.g., 10-fold or more, or 100-fold or more decrease in expression) or lack of detectable expression of guaB.

In some embodiments, the method of the invention results in increased plasmid production, including supercoiled plasmids, in a host cell. Without wishing to be bound by theory, the increased plasmid production or increased supercoiled plasmid production or yield, may be due to higher expression (e.g., overexpression of the guaB gene in relation to its expression in the wild type guaB+ host under the same conditions). As discussed above, the guaB gene encodes IMPDH, which is a rate-limiting enzyme in the production of guanine nucleotides and thus, an important regulator of cell proliferation. Over expression of IMPDH may overcome a rate limiting bottleneck that affects the amount of plasmid DNA, including supercoiled plasmid DNA, that can be produced in a cell. The level of expression of the guaB gene can be controlled in a host cell by incorporating one or more copies of the guaB gene on the vector, and under control of any of various promoters known in the art that provide variable levels of gene expression. Alternatively, the one or more copies of the guaB gene may be supplied to the host cell by a vector separate from, but preferably compatible with, the plasmid or vector of interest. Methods of the invention involving overexpression of the guaB gene to increase plasmid production can be applied in connection with any host cell line suitable for recombinant gene expression, including, but not limited to, mammalian cell lines, insect cell lines, yeast cell lines, and bacterial cell lines. In these embodiments of the invention, the host may be a guaB+ host, and the vector or plasmid of interest may alternatively employ an antibiotic resistance marker, since the purpose of the guaB gene in such embodiments is to increase plasmid yield. Preferably, however, the plasmid of interest does not contain an antibiotic resistance gene.

Thus, in a related embodiment, the invention provides a method for increasing plasmid copy number in a host cell, or for increasing the yield of purified plasmid, including supercoiled plasmid, comprising transforming the host cell with a plasmid of interest, and optionally with a second vector. The plasmid of interest and/or the second vector comprises one or more copies of a guaB gene under control of one or more promoters, suitable for increasing the level of expression of guaB (e.g., overexpressing guaB), or the IMPDH polypeptide, in the host cell. Purification of the plasmid of interest may therefore provide higher plasmid yields. In some embodiments, the plasmid of interest overexpresses guaB, and does not contain an antibiotic resistance marker. In these embodiments, the host cell may be a guaB− host cell as described above, such that the plasmid of interest not only provides for selection via guaB complementation, but also provides for overexpression of guaB to support higher plasmid yields.

The guaB gene suitable for complementing a guaB− host, or for supporting an increased plasmid yield, in accordance with the invention, may be any gene that encodes an IMPDH, including bacterial and yeast genes. In one embodiment, the guaB gene is an E. coli guaB gene (e.g., SEQ ID NO:1). The protein product of the guaB gene may further be at least 70%, at least 80%, at least 90%, or at least 95% (or identical) to E. coli IMPDH (SEQ ID NO: 2), but in each case retains IMPDH activity required for GTP synthesis.

Vectors suitable in this aspect of the invention are described below.

### Vectors and Expression Systems

A vector is a composition for delivering a polynucleotide of interest to the interior of a cell. Numerous vectors are known in the art including, but not limited to, linear polynucleotides, transposons, polynucleotides associated with ionic or amphiphilic compounds, plasmids, bacteriophages and viruses. Examples of viral vectors include, but are not limited to, adenoviral vectors, adeno-associated virus vectors, retroviral vectors, and the like.

In one aspect, the invention comprises a vector containing a functional guaB gene, or a gene encoding an IMPDH, or other gene involved in the cellular production of GTP in recombinant host microorganisms. The vector may further comprise at least one polynucleotide sequence of interest, or may contain convenient restriction sites for insertion of such a sequence to be operably controlled by a promoter. In some embodiments, the vector does not contain a functional antibiotic resistance gene, or does not encode or express gene products necessary for imparting antibiotic resistance to a host microorganism. Thus, the vector may be sufficient for complementing a host microorganism's guaB deficiency or inactivation. In accordance with this aspect of the invention, the presence of an antibiotic selection marker on the vector is unnecessary, making the vector desirable for pharmaceutical applications particularly.

In one embodiment, the vector is a plasmid, including plasmids designed to integrate into the host chromosome, and plasmids that are autonomously replicating in the host cell. The plasmid may be maintained in the host cell at various levels, and therefore, the vector of the invention may be a low copy-number plasmid or high copy-number plasmid. Alternatively, the vector may be a viral vector.

The vectors of the invention may lack a functional antibiotic resistance gene, or lack such a gene altogether, but express a guaB gene, or a gene encoding an IMPDH polypeptide, or other gene involved in the cellular production of GTP in recombinant host microorganisms, so as to allow their selection in a host cell as described above. The host cell can be engineered by deletion or inactivation of the guaB gene, or alternatively, the host cell may be naturally deficient in the guaB gene, or gene product. Exemplary host cells that can be engineered for use with the invention are bacteria, including Escherichia coli, Bacillus subtilis, and Salmonella; as well as yeasts: including Sacharromyces cerevisiae, Candida albicans and Schizosaccharomyces pombe, and Pichia pastoris. In some embodiments, the host microorganism harbors an additional mutation that will help increase the retention or copy number of the plasmids or vectors of the invention that replicate in the microorganism. For example, the microorganism may further have a recA mutation or an endA mutation, or both.

In a related aspect, the invention provides for an expression system comprising a host microorganism containing an inactive or substantially inactive guaB gene (as described), and a vector of the invention carrying a recombinant gene, or suitable for carrying a recombinant gene, and for complementing the inactivation or substantial inactivation of guaB. Such systems may take the form of a kit providing the microorganism and vector, e.g., as packaged for commercial sale. Such kits find use as laboratory tools for producing products (e.g., microoganisms, plasmids, expression products) without involving selection by antibiotic resistance.

Alternatively, the expression system provides for a microorganism that expresses a product of interest from the vector (e.g., an RNA or protein, e.g. as described herein). Such microorganisms (expressing a product of interest) are optionally suitable for administration to a mammal in need of a particular treatment, and provide a convenient mechanism for delivering the product of interest to a subject or patient for therapy. In accordance with the invention, such microorganisms can be isolated during production by guaB complementation, and without selection for antibiotic resistance, which would be undesirable where the microorganism is to be administered to a patient. In accordance with this embodiment, the microorganism is a live, optionally attenuated and/or invasive, but generally or substantially non-infectious, microorganism. Such microorganisms include known species and/or stains of E. coli, Salmonella, and Listeria. The microorganism may be administered to a subject, for example, by contact with mucosal surfaces of the subject (including the GI tract or genitourinary tract), or by contact with skin, so as to be taken up by host cells (e.g., including phagocytic cells), thereby delivering the product of interest to the host cells. The product of interest may be a siRNA (e.g., shRNA), as described herein, for silencing a gene in a host cell. Such a system, providing a microorganism (e.g., E. coli) containing an antibiotic resistance gene-free vector encoding RNAs such as shRNAs having therapeutic utility against mammalian disease-related genes as e.g., the colon-cancer causing beta-catenin gene or the E6 and/or E7 gene of human papilloma virus (HPV) types 16 and 18, may be provided in a pharmaceutically acceptable carrier for delivery to the relevant human cells as described herein.

General texts which describe molecular biological techniques, which are applicable to the present invention, such as cloning, mutation, cell culture and the like, include Berger and Kimmel, Guide to Molecular Cloning Techniques, Methods in Enzymology volume 152 Academic Press, Inc., San Diego, Calif. (Berger); Sambrook et al., Molecular Cloning—A Laboratory Manual (3rd Ed.), Vol. 1-3, Cold Spring Harbor Laboratory, Cold Spring Harbor, N.Y., 2000 (“Sambrook”) and Current Protocols in Molecular Biology, F. M. Ausubel et al., eds., Current Protocols, a joint venture between Greene Publishing Associates, Inc. and John Wiley & Sons, Inc., (“Ausubel”). These texts describe mutagenesis, the use of vectors, promoters and many other relevant topics related to, e.g., the engineering or mutagenesis of microorganisms etc. Thus, the invention may involve known methods of protein engineering and recombinant DNA technology to mutagenize and/or knockout (via deletion and/or insertion) the guaB gene in a host microorganism of choice. Various types of mutagenesis techniques can be utilized to modify/mutate the guaB gene in a microorganism. They include but are not limited to site-directed mutagenesis, random point mutagenesis, homologous recombination (DNA shuffling), oligonucleotide-directed mutagenesis, phosphorothioate-modified DNA mutagenesis, mutagenesis using gapped duplex DNA or the like.

In some embodiments, the vectors of the invention may be prepared by removing an antibiotic resistance gene that may be present, and/or adding a guaB gene, or other gene suitable for complementing a deficiency in synthesis of GTP. Virus vectors, such as baculovirus, poxvirus (e.g., vaccinia virus, avipox virus, canarypox virus, fowlpox virus, raccoonpox virus, swinepox virus, etc.), adenovirus (e.g., canine adenovirus), herpesvirus, and retrovirus can be modified accordingly. Other vectors that may be so modified, include vectors for use in bacteria, such as pQE70, pQE60 and pQE-9, pBluescript vectors, Phagescript vectors, pNH8A, pNH16a, pNH18A, pNH46A, ptrc99a, pKK223-3, pKK233-3, pDR540, pRIT5. Among suitable eukaryotic vectors are pFastBac1 pWINEO, pSV2CAT, pOG44, pXT1 and pSG, pSVK3, pBPV, pMSG, and pSVL. Other suitable vectors will be readily apparent to the skilled artisan.

In one embodiment, the vectors described in copending applications WO 2005/014806, WO 2006/033756, International Application No. PCT/US2008/60222 entitled “Influenza Polynucleotides, Expression Constructs, Compositions, and Methods of Use” and International Application No. PCT/US2007/81103 entitled “MicroRNA-Formated Multitarget Interfering RNA Vector Constructs and Methods of Using the Same,” which are hereby incorporated by reference, can be modified in accordance with the invention. For example, vectors may be designed so as to express products of interest from bacterial promoters, and/or to contain a guaB gene (as described herein), and to not contain an antibiotic-resistance gene.

The vectors of the present invention find use as DNA vaccines, for example encoding a protein antigen. Thus, such a DNA vaccine is an antibiotic resistance gene-free plasmid comprising: a first nucleic acid sequence encoding a polypeptide antigen; and a functional guaB gene. The antibiotic resistance gene-free plasmid may be grown in a guaB deficient microorganism, as described. In these or other embodiments, the DNA vaccine is an antibiotic resistance gene-free plasmid comprising: a first nucleic acid sequence encoding a polynucleotide that enhances the immune response of an animal, and a functional guaB gene. The antibiotic resistance gene-free plasmid may be administered as purified plasmid using a suitable transfection composition for administering DNA to a subject, or alternatively, may be administered with a microorganism to express the gene product, as described above. For example, the plasmid may be administered via a live microorganism, which is optionally attenuated and/or invasive, but generally or substantially non-infectious. Such microorganisms include known species and/or strains of E. coli, Salmonella, and Listeria, among others.

In another embodiment, vectors of the present invention comprise a promoter/regulatory sequence operably linked to the gene of interest, such as a gene encoding an antigen, an siRNA, shRNA, the guaB gene, or a combination thereof. In another embodiment, the plasmid of the present invention is replicated and propagated in a prokaryotic or eukaryotic host, such as a bacterium, and administered to an animal, e.g. a human. Therefore, in some embodiments, the vector of the present invention comprises both eukaryotic and prokaryotic expression or regulatory elements (e.g., promoters). For example, the vector may comprise prokaryotic promoter/regulatory elements including but are not limited to, T7, SP60, trp operon, tRNA promoters, lac operon, recA, lexA, and the like. Prokaryotic promoters are well known in the art, as are methods for their use. In some embodiments, the vector comprises a prokaryotic promoter operably linked to a guaB gene.

Vectors of the present invention comprise, or further comprise, a eukaryotic promoter. A eukaryotic promoter useful in the present invention include constitutive, inducible, or tissue-specific promoters. Many promoter/regulatory sequences useful for driving constitutive expression of a gene are available in the art and include, but are not limited to, for example, the cytomegalovirus immediate early promoter enhancer sequence, the SV40 early promoter, the immunoglobulin promoter, as well as the Rous sarcoma virus promoter, and the like. Moreover, inducible and tissue specific expression of an RNA, antigen, or enzyme can be accomplished by placing the nucleic acid encoding such a molecule under the control of an inducible or tissue specific promoter/regulatory sequence. Examples of tissue specific or inducible promoter/regulatory sequences which are useful for this purpose include, but are not limited to, the MMTV LTR inducible promoter and the SV40 late enhancer/promoter. In addition, promoters which are well known in the art to be induced in response to inducing agents such as metals, glucocorticoids, tetracycline, hormones, and the like, are also contemplated for use with the invention. Thus, it will be appreciated that the invention includes the use of any promoter/regulatory sequence known in the art that is capable of driving expression of the desired protein operably linked thereto. For expression of short transcripts of 400 to 500 nucleotides or less, polymerase III promoters, especially U6-type polymerase III promoters such as H1, U6, and 7SK, are especially useful. A preferred 7SK promoter is the 7SK A4 promoter variant taught in WO 06/033756, the nucleotide sequence of which is hereby incorporated by reference.

In certain embodiments, the present invention comprises an antibiotic resistance gene-free vector (e.g., an expression construct) containing a DNA segment that encodes an expressed interfering RNA (eiRNA) molecule, with the DNA segment being operably linked to a promoter to drive expression of the RNA molecule. An “expression construct” is any double-stranded DNA or double-stranded RNA designed to direct production of an RNA of interest. For example, the construct may contain at least one promoter that is operably linked to a gene, coding region, or polynucleotide sequence of interest. A polynucleotide sequence of interest may be: a cDNA or genomic DNA fragment, either protein encoding or non-encoding; an RNA effector molecule such as an antisense RNA, triplex-forming RNA, ribozyme, an artificially selected high affinity RNA ligand (aptamer); a double-stranded RNA, e.g., an RNA molecule comprising a stem-loop or hairpin dsRNA, or a bi-finger or multi-finger dsRNA or a microRNA, or any RNA of interest. The invention includes expression constructs in which one or more of the promoters is not in fact operably linked to a polynucleotide sequence to be transcribed, but instead is designed for efficient insertion of an operably-linked polynucleotide sequence to be transcribed by the promoter, for instance by way of one or more restriction cloning sites in operative association with the one or more promoters. Examples of eiRNA comprise microRNA (miRNA, see copending International Application No. PCT/US2007/81103 entitled “MicroRNA-Formated Multitarget Interfering RNA Vector Constructs and Methods of Using the Same”, herein incorporated by reference in its entirety), short interfering (siRNA) and short hairpin RNA (shRNA).

Transfection or transformation of the vector of the invention into a recipient cell allows the cell to express an RNA effector molecule encoded by the vector or expression construct. The recipient cell may be a microorganism, or may be an animal, e.g. a human, cell. An expression construct may be a genetically engineered plasmid, virus, recombinant virus, or an artificial chromosome derived from, for example, a bacteriophage, adenovirus, adeno-associated virus, retrovirus, lentivirus, poxvirus, or herpesvirus.

The vectors or expression constructs of the present invention may be used to target viral sequences, for example, by RNAi-mediated gene silencing. Exemplary viruses that may be targeted by the vectors of the present invention include but are not limited to Retroviridae (e.g. human immunodeficiency viruses, such as HIV-1 (also referred to as HTLV-III, LAV or HTLV-III/LAV, or HIV-III; and other isolates, such as HIV-LP)); Picornaviridae (e.g. polio viruses, hepatitis A virus; enteroviruses, human coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronaviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses: avian and seasonal, see copending International Application No. PCT/US2008/60222 entitled “Influenza Polynucleotides, Expression Constucts, Compositions, and Methods of Use”, incorporated herein by reference in its entirety for all purposes); Bungaviridae (e.g. Hantaan viruses, bunga viruses, phleboviruses and Nairo viruses); Arena viridae (hemorrhagic fever viruses); Reoviridae (e.g. reoviruses, orbiviruses and rotaviruses); Birnaviridae; Hepadnaviridae (Hepatitis B virus, Hepatitis C virus, see WO 2005/014806 and WO 2006/069064, incorporated herein by reference in their entireties for all purposes); Parvovirida (parvoviruses); Papovaviridae (papilloma viruses, including human papillomavirus (HPV) such as HPV 16 and HPV 18, polyoma viruses); Adenoviridae (most adenoviruses); Herpesviridae (herpes simplex virus (HSV) 1 and 2, varicella zoster virus, cytomegalovirus (CMV), herpes virus); Poxyiridae (variola viruses, vaccinia viruses, pox viruses); and Iridoviridae (e.g. African swine fever virus); and unclassified viruses (e.g. the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

The vectors of the invention which encode eiRNA can be used for the treatment or prevention of an autoimmune disease. An autoimmune disease is the result of an inappropriate and excessive response to a self-antigen. Examples of autoimmune diseases, include but are not limited to, Addision's disease, alopecia greata, ankylosing spondylitis, autoimmune hepatitis, autoimmune parotitis, Crohn's disease, diabetes (Type I), dystrophic epidermolysis bullosa, epididymitis, glomerulonephritis, Graves' disease, Guillain-Barr syndrome, Hashimoto's disease, hemolytic anemia, systemic lupus erythematosus, multiple sclerosis, myasthenia gravis, pemphigus vulgaris, psoriasis, rheumatic fever, rheumatoid arthritis, sarcoidosis, scleroderma, Sjogren's syndrome, spondyloarthropathies, thyroiditis, vasculitis, vitiligo, myxedema, pernicious anemia, ulcerative colitis, among others.

The vectors of the invention that comprise eiRNA can be used for the treatment or prevention of cancer. The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

Additional products of interest that may be delivered, include siRNAs (including shRNAs) for targeting beta-catenin RNA. Targeting beta-catenin RNA via gene silencing is a particularly useful therapy for patients with familial adenomatous polyposis (FAP). Other products of interest may target genes involved in the symptoms or progression of Inflammatory Bowel Diseases (IBD), many of which are known in the art.

The vector of the invention comprising an expression construct may be engineered to encode multiple, e.g., three, four, five or more RNA molecules, such as short hairpin dsRNAs and/or other RNAs. The encoded RNAs may be separate, or in the form of bi-finger or multi-finger constructs comprising hairpin or stem loop regions according to the invention separated by a single-stranded region of at least about 5, 10, 15, 20, or 25 nucleotides or more. See application nos. WO 2000/63364 and WO 2004/035765, which are hereby incorporated by reference in their entireties.

In certain embodiments, the vector of the invention comprising an expression construct encodes two or more eiRNAs, such as 2, 3, 4, 5, or more eiRNA molecules, e.g. double stranded RNA (dsRNA). The construct may further encode eiRNAs as double-stranded hairpin molecules.

Where it is desired to deliver short eiRNAs, multiple RNA polymerase III promoter expression constructs (as taught in WO 06/033756, which is hereby incorporated by reference), may be used in accordance with the invention. The multiple RNA polymerase III promoters may be utilized in conjunction with promoters of other classes, including RNA polymerase I promoters, RNA polymerase II promoters, etc. Preferred in some applications are the Type III RNA pol III promoters including U6, H1, and 7SK, which exist in the 5′ flanking region, include TATA boxes, and lack internal promoter sequences. A preferred 7SK promoter is the 7SK 4A promoter variant taught in WO 06/033756, the nucleotide sequence of which is hereby incorporated by reference. In such expression constructs each promoter may be designed to control expression of an independent RNA expression cassette, e.g., a shRNA expression cassette. RNA Pol III promoters may be especially beneficial for expression of small engineered RNA transcripts, because RNA Pol III termination occurs efficiently and precisely at a short run of thymine residues in the DNA coding strand, without other protein factors. T₄ and T₅ are the shortest Pol III termination signals in yeast and mammals, with oligo (dT) terminators longer than T₅ being rare in mammals. Accordingly, the multiple polymerase III promoter expression constructs of the invention will include an appropriate oligo (dT) termination signal, i.e., a sequence of 4, 5, 6 or more Ts, operably linked 3′ to each RNA Pol III promoter in the DNA coding strand. A DNA sequence encoding an RNA effector molecule, e.g., a dsRNA hairpin or RNA stem-loop structure to be transcribed, is inserted between the Pol III promoter and the termination signal.

The invention provides means for delivering to a host cell sustained amounts of 2, 3, 4, 5, or more different dsRNA hairpin molecules (e.g., specific for 2, 3, 4, 5, or more different viral sequence elements), in a genetically stable mode, so as to inhibit viral replication without evoking a dsRNA stress response. In accordance with these embodiments, each dsRNA hairpin may be expressed from an expression construct, and controlled by an RNA polymerase III promoter. Other promoters may also be used, including, but not limited to, RNA pol I and pol II promoters.

Thus, vectors of the invention comprising expression constructs provide a convenient means for delivering a multi-drug regimen comprising several different RNAs to a cell or tissue of a host vertebrate organism, without introducing an antibiotic resistance gene.

Methods of growing the engineered microorganisms comprising the vectors of the invention are also contemplated as part of the invention. Methods of growing the engineered microorganisms include, but are not limited to, batch, batch-fed, continuous and perfusion cell culture techniques. Cell culture includes the growth and propagation of microorganisms in a bioreactor (a fermentation chamber) wherein vectors of the invention propagate and can eventually be isolated and purified. Typically, cell culture is performed under sterile, controlled temperature and atmospheric conditions in a bioreactor. A bioreactor is a chamber used to culture cells in which environmental conditions such as temperature, atmosphere, agitation and/or pH can be monitored. In one embodiment, the bioreactor is a stainless steel chamber. In another embodiment, the bioreactor is a pre-sterilized plastic bag (e.g. Cellbag®, Wave Biotech, Bridgewater, N.J.). In another embodiment, the pre-sterilized plastic bags are about 50 L to 1000 L bags.

The media used to grow the cells of the invention should not contain guanine. As stated above a guaB⁻ microorganism, which does not carry the guaB plasmid, does not grow well in medium without guanine. Thus, in order to increase the selective pressure for microorganisms to retain the vectors of the invention, the microorganisms should be grown in media without guanine, or without amounts of guanine suitable to support growth of the guaB− host. Defined media and minimal media that can be used to conduct the methods of the invention are well known in the art.

In some embodiments, the vector of the invention will be isolated and purified from the organism. Methods of purifying vectors, e.g. plasmids are known in the art.

### Pharmaceutical Compositions

Generally, the vectors of the invention do not contain an antibiotic resistance gene, or a functional antibiotic resistance gene, since such genes are undesirable for vectors having human or veterinary applications. The vectors of the invention are, therefore, useful in gene therapy applications and/or DNA vaccine applications and/or eiRNA applications. The invention provides a pharmaceutical composition comprising a vector of the invention, and a DNA delivery vehicle. The pharmaceutical compositions may be administered to a cell or an animal, to deliver a dose effective for delivering the vector of the invention to the desired cells. The pharmaceutical compositions may employ the delivery vehicles described in U.S. application Ser. No. 10/513,708, which is hereby incorporated by reference in its entirety. For example, exemplary compositions may employ polycationic molecules, such as spermine or spermadine, and/or may employ endosomolytic molecules (e.g., such as cholesterol) to enhance transfection of host cells. In some embodiments, an antibiotic-resistance-gene-free expression vector of the invention may be complexed with a cationic amphiphile such as the local anaesthetic bupivacaine or another transfection facilitating compound known to those of skill in the art.

The pharmaceutical compositions of the invention may be formulated in accordance with conventions in the art. In general, such preparatory methods include the step of bringing the active ingredient into association with a carrier or one or more other accessory ingredients, and then, if necessary or desirable, shaping or packaging the product into a desired single- or multi-dose unit. Suitable carriers include, but are not limited to, saline, buffered saline, dextrose, water, glycerol, ethanol, and combinations thereof. The composition can be adapted for the mode of administration and can be in the form of, for example, aerosol, powder, or liquid.

Where the composition is formulated as a microorganism (e.g., a guaB− host expressing a product of interest from a vector further containing a guaB-complementing gene), the composition is preferably formulated for delivery to the GI tract or genitourinary tract.

Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

In some embodiments, the compositions of the invention (e.g., containing purified vector) may be administered via parenteral administration, which includes physical breaching of a tissue of a subject and administration of the pharmaceutical composition through the breach in the tissue. Parenteral administration may take the form of injection of the composition, e.g., by application of the composition through a surgical incision, or by application of the composition through a tissue-penetrating non-surgical wound, and the like. In particular, parenteral administration is contemplated to include, but is not limited to, subcutaneous, intraperitoneal, intramuscular, intrasternal injection, intravenous (IV), intra-arterial, intradermal, intrathecal, intramuscular (IM) and kidney dialytic infusion techniques. The compositions of the invention may also be administered, without limitation, topically, orally, and by mucosal routes of delivery such as intranasal, inhalation, rectal, vaginal, buccal, and sublingual.

The present invention may, in certain embodiments, employ the methods disclosed in the U.S. Provisional Application No. 60/907,014, filed Mar. 16, 2007 entitled “Methods and Compositions For Directing RNAi-Mediated Gene Silencing In Distal Organs Upon Intramuscular Administration Of DNA Expression Vectors,” which is hereby incorporated by reference in its entirety. Specifically, intramuscular injection or electroporation of expression constructs encoding dsRNA(s) may result in targeted inhibition of gene expression in other organs and tissues of the body.

The following examples are provided to describe and illustrate the present invention. As such, they should not be construed to limit the scope of the invention. Those in the art will well appreciate that many other embodiments also fall within the scope of the invention, as it is described hereinabove and in the claims.

### EXAMPLES

### Example 1

### Construction of IMPDH shRNA Vector

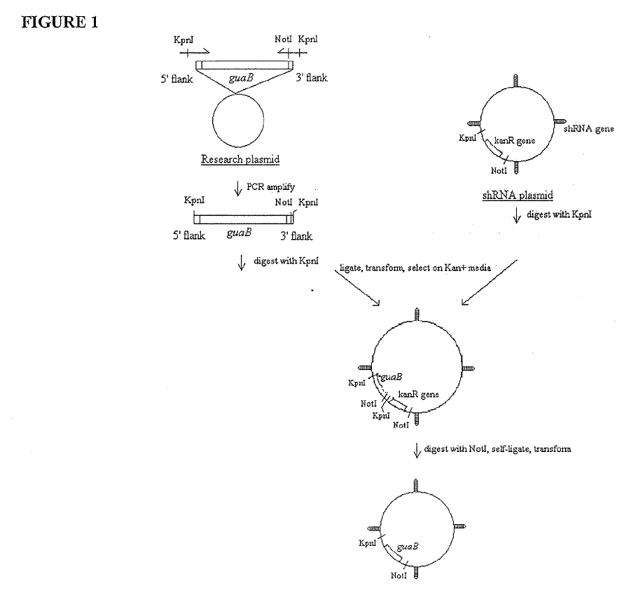

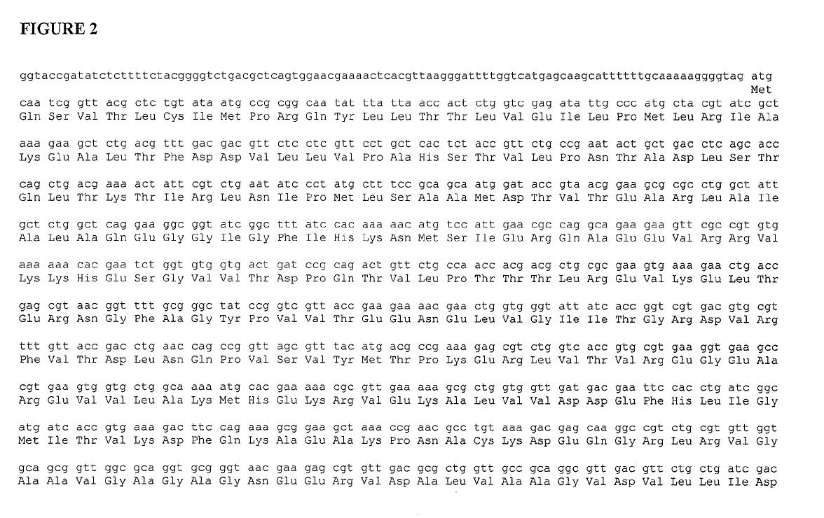

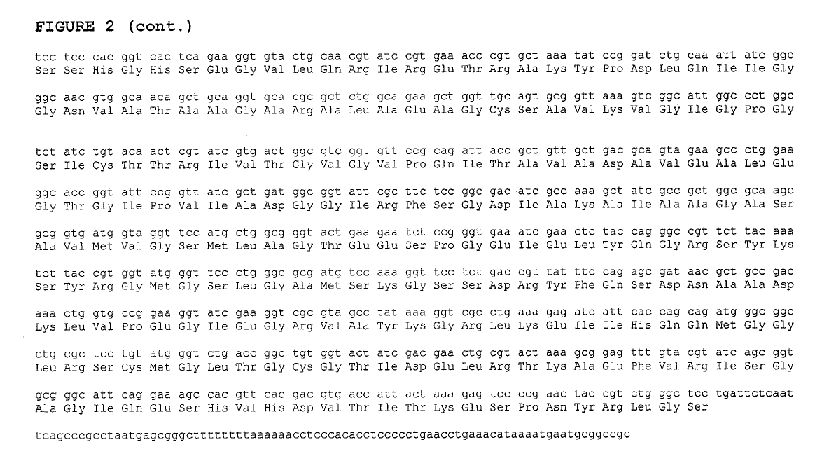

The E. coli guaB gene (encoding the IMP dehydrogenase (IMPDH) protein) (GenBank accession number X02209, which is hereby incorporated by reference), along with non-guaB 5′ and 3′ flanking regions important for expression of this gene in E. coli, was amplified by PCR from a research plasmid containing the gene and flanking regions (see FIG. 1 for cloning strategy and FIG. 2 for sequence). The primers used to amplify the guaB+flanking regions contained a KpnI restriction endonuclease (RE) site on the forward primer and NotI and KpnI RE sites on the reverse primer. Thus, the guaB+flanking region PCR product had a KpnI site on the 5′ end and NotI and KpnI sites on the 3′ end. The guaB+flanking region PCR product was digested with KpnI and gel-purified.

A plasmid vector containing the genes for four short-hairpin RNAs and containing a kanamycin resistance gene with a KpnI RE site at its 3′ side and a NotI site its 5′ end was used for the insertion of the guaB+flanking regions. This vector was digested with KpnI and the guaB+flanking region KpnI-digested PCR was ligated into the KpnI site. The ligation product was transformed into E. coli and selected on solid bacterial media containing kanamycin. Colonies derived from clones containing the guaB+flanking regions were analyzed and confirmed for the presence of the guaB insert by restriction analysis. The kanamycin resistance gene was then removed by digestion of one of these clones with NotI and then re(self)-ligation. Confirmation of the presence of the guaB gene was accomplished by transforming into a guaB⁻ E. coli host in liquid growth media devoid of guanine. The presence of the guaB gene (and absence of the kanamycin-resistance gene) was additionally confirmed by nucleotide sequence analysis.

### Example 2

### Deletion of guaB in E. coli by a FRT-Flanked Kan-Cassette

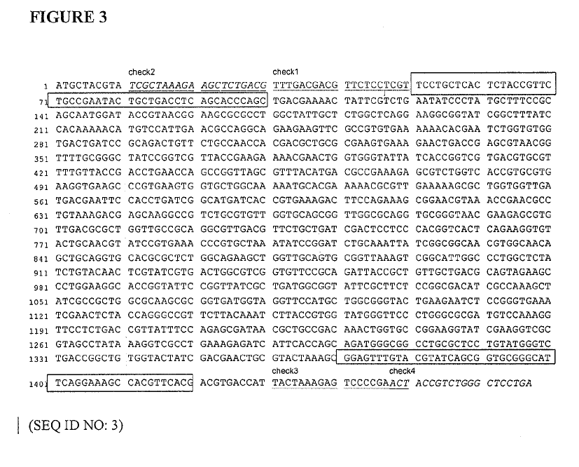

A FRT-flanked Kan-cassette was amplified by PCR using long oligos containing the homology-arm sequences (see FIG. 3).

The PCR was performed with the following:

| | |
|---|---|
| template Kan-cassette | 1 μL |
| dNTPs (5 mM; Eppendorf) | 3 μL |
| Primer “guaB up” (10 μM; BioSpring) | 1 μL |
| Primer “guaB down” (10 μM; BioSpring) | 1 μL |
| 5× HF Buffer (Finnzyme) | 10 μL |
| Phusion (Finnzyme) | 0.5 μL |
| H₂O | 33.5 μL |

Next, the PCR products where analyzed on an agarose gel. The fragment showed the correct size and was used for Red/ET recombination.

A single colony of both strains harbouring plasmid pRed/ET was picked and grown O/N at 30° C. in 1 ml medium containing tetracycline (3 μg/ml). The next day a fresh culture was incubated in LB at 30° C. and at OD650 0.2 the Red/ET recombination proteins were induced by addition of 10% L-arabinose, keeping control cells uninduced. The L-arabinose treated bacterial cells were electroporated with 2 μL of 600 ng/uL PCR-product at 1350V (5 mS). The cells were resuspended in 1 mL ice-cold LB-medium and shaken with 1050 rpm at 37° C. for 180 min. The whole culture was centrifuged, resuspended and plated on plates containing kanamycin (50 μg/ml) and incubated 0/N at 37° C. The ratio of colonies of induced to non-induced on the plates was several hundreds to zero.

Single colonies were purified and the correct insertion of the Kan-cassette was verified by PCR.

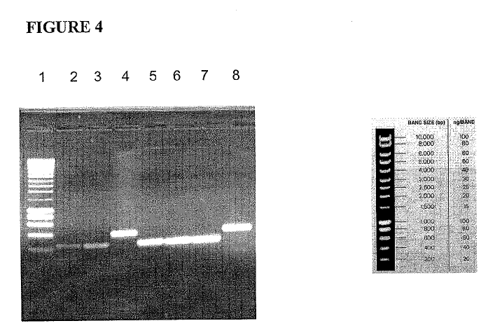

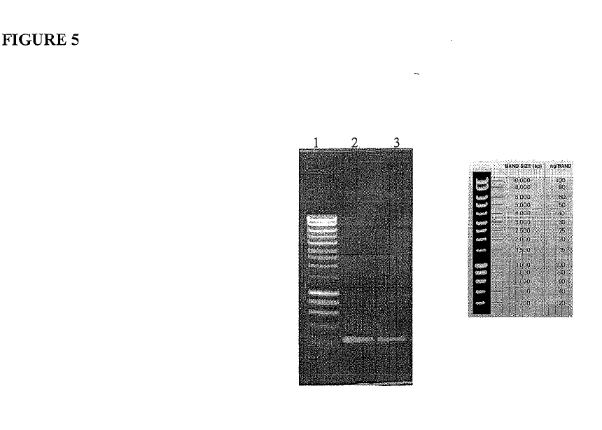

The check PCR was performed by using primers located on the chromosome and primers located in the cassette and showed the expected results (see FIG. 4).

The next step was the removal of the antibiotic cassettes by a FLP-recombinase step. Therefore, plasmid Flp706 carrying FLP-recombinase and a Tet-resistance gene was transformed into the triple and quadruple mutant and selected on LB Kan Tet plates.

A single colony was picked and incubated overnight in LB-medium containing tetracycline at 30° C. 20 μl of the grown culture were incubated in LB at 30° C. for 2 h. Cre-Recombinase expression is switched on by a temperature shift to 37° C. and incubation O/N at 37° C. At this temperature Flp706 gets lost. A little bit of the overnight culture was streaked out with a loop on LB plates and incubated overnight. 24 single colonies of both mutants were analyzed in a marker test. The Kan-sensitive strains were analyzed by PCR (see FIG. 4). The colony showing the correct construct was preserved for future use.

All publications and patent applications herein are incorporated by reference to the same extent as if each individual publication or patent application was specifically and individually indicated to be incorporated by reference.

The foregoing detailed description has been given for clearness of understanding only and no unnecessary limitations should be understood there from as modifications will be obvious to those skilled in the art. It is not an admission that any of the information provided herein is prior art or relevant to the presently claimed inventions, or that any publication specifically or implicitly referenced is prior art.

Unless defined otherwise, all technical and scientific terms used herein have the same meaning as commonly understood by one of ordinary skill in the art to which this invention belongs.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

1. A method for selecting recombinant microorganisms, comprising: a) transforming a microorganism comprising an inactive or deleted guaB gene with a vector comprising a complementing guaB gene; and b) growing said transformed microorganism on media lacking sufficient guanine, so as to permit selective growth of transformed microorganisms.

2. The method of claim 1, wherein the microorganism has a knockout of guaB.

3. The method of claim 1, wherein said microorganism has a mutation, deletion or insertion at guaB.

4. The method of claim 1, wherein the vector contains a polynucleotide sequence molecule operably linked to a promoter.

5. A method for increasing plasmid yield, comprising: transforming a host cell with a plasmid of interest, and optionally with a second vector, wherein the plasmid of interest and/or the second vector comprise one or more copies of a guaB gene under control of one or more promoters, so as to increase the level of expression of guaB in the host cell.

6. The method of claim 5, wherein said vector is a plasmid or a viral vector.

7. The method of claim 6, wherein said vector integrates into a host chromosome.

8. The method of claim 6, wherein said plasmid is an autonomously replicating plasmid.

9. The method of claim 8, wherein said plasmid is a low copy-number plasmid.

10. The method of claim 8, wherein said plasmid is a high copy-number plasmid.

11. The method of claim 5, wherein the plasmid of interest contains a polynucleotide sequence operably linked to a promoter.

12. The method of claim 11, wherein the plasmid of interest overexpresses guaB in the host, and complements the host's guaB deficiency.

13. The method of claim 1, wherein the microorganism is selected from the group consisting of a bacteria and a yeast.

14. The method of claim 13, wherein said bacteria is selected from the group consisting of Escherichia coli, Bacillus subtilis, and Salmonella.

15. The method of claim 13, wherein said yeast is selected from the group consisting of Sacharromyces cerevisiae, Candida albicans and Schizosaccharomyces pombe, and Pichia pastoris.

16. A vector comprising a guaB gene and at least one polynucleotide sequence encoding a macromolecule of interest, wherein said vector does not contain an antibiotic resistance gene.

17. The vector of claim 16, wherein said vector is a plasmid or a viral vector.

18. The vector of claim 16, wherein said plasmid integrates into a host chromosome.

19. The vector of claim 17, wherein said plasmid is an autonomously replicating plasmid.

20. The vector of claim 17, wherein said plasmid is a low copy-number plasmid.

21. The vector of claim 17, wherein said plasmid is a high copy-number plasmid.

22. The vector of claim 16, wherein said macromolecule of interest encodes an eiRNA, a shRNA, or a protein.

23. The vector of claim 16, wherein said vector can complement a microorganism having a guaB inactivation or deletion, and is sufficient to support growth on minimal media lacking guanine.

24. The vector of claim 16, wherein the vector is suitable for replication in a bacteria or yeast.

25. The vector of claim 24, wherein said bacteria is selected from the group consisting of Escherichia coli, Bacillus subtilis, and Salmonella.

26. The vector of claim 24, wherein said yeast is selected from the group consisting of Sacharromyces cerevisiae, Candida albicans and Schizosaccharomyces pombe, and Pichia pastoris.

27. The vector of claim 16, wherein said vector is contained within a substantially noninfectious microorganism that expresses the macromolecule of interest.

28. The vector of claim 27, wherein said substantially non-infectious microorganism has a guaB− genotype, said guaB− genotype being complemented by said vector.

29. A pharmaceutical composition comprising the vector of claim 16.

30. An expression system comprising a guaB⁻ host and the vector of claim 16.


SEQUENCE LISTING (3 sequences)

SEQ ID NO: 1 - DNA, 1730 bp, Escherichia coli
  CDS (103)..(1638)
ggtaccgatatctcttttctacggggtctgacgctcagtggaacgaaaactcacgttaagggattttggtcatgagcaagcattttttgcaaaaaggggtagatgcaatcggttacgctctgtataatgccgcggcaatatttattaaccactctggtcgagatattgcccatgctacgtatcgctaaagaagctctgacgtttgacgacgttctcctcgttcctgctcactctaccgttctgccgaatactgctgacctcagcacccagctgacgaaaactattcgtctgaatatccctatgctttccgcagcaatggataccgtaacggaagcgcgcctggctattgctctggctcaggaaggcggtatcggctttatccacaaaaacatgtccattgaacgccaggcagaagaagttcgccgtgtgaaaaaacacgaatctggtgtggtgactgatccgcagactgttctgccaaccacgacgctgcgcgaagtgaaagaactgaccgagcgtaacggttttgcgggctatccggtcgttaccgaagaaaacgaactggtgggtattatcaccggtcgtgacgtgcgttttgttaccgacctgaaccagccggttagcgtttacatgacgccgaaagagcgtctggtcaccgtgcgtgaaggtgaagcccgtgaagtggtgctggcaaaaatgcacgaaaaacgcgttgaaaaagcgctggtggttgatgacgaattccacctgatcggcatgatcaccgtgaaagacttccagaaagcggaagctaaaccgaacgcctgtaaagacgagcaaggccgtctgcgtgttggtgcagcggttggcgcaggtgcgggtaacgaagagcgtgttgacgcgctggttgccgcaggcgttgacgttctgctgatcgactcctcccacggtcactcagaaggtgtactgcaacgtatccgtgaaacccgtgctaaatatccggatctgcaaattatcggcggcaacgtggcaacagctgcaggtgcacgcgctctggcagaagctggttgcagtgcggttaaagtcggcattggccctggctctatctgtacaactcgtatcgtgactggcgtcggtgttccgcagattaccgctgttgctgacgcagtagaagccctggaaggcaccggtattccggttatcgctgatggcggtattcgcttctccggcgacatcgccaaagctatcgccgctggcgcaagcgcggtgatggtaggttccatgctggcgggtactgaagaatctccgggtgaaatcgaactctaccagggccgttcttacaaatcttaccgtggtatgggttccctgggcgcgatgtccaaaggttcctctgaccgttatttccagagcgataacgctgccgacaaactggtgccggaaggtatcgaaggtcgcgtagcctataaaggtcgcctgaaagagatcattcaccagcagatgggcggcctgcgctcctgtatgggtctgaccggctgtggtactatcgacgaactgcgtactaaagcggagtttgtacgtatcagcggtgcgggcattcaggaaagccacgttcacgacgtgaccattactaaagagtccccgaactaccgtctgggctcctgattctcaattcagcccgcctaatgagcgggcttttttttaaaaaacctcccacacctccccctgaacctgaaacataaaatgaatgcggccgc

SEQ ID NO: 2 - protein, 511 aa, Escherichia coli
MQSVTLCIMPRQYLLTTLVEILPMLRIAKEALTFDDVLLVPAHSTVLPNTADLSTQLTKTIRLNIPMLSAAMDTVTEARLAIALAQEGGIGFIHKNMSIERQAEEVRRVKKHESGVVTDPQTVLPTTTLREVKELTERNGFAGYPVVTEENELVGIITGRDVRFVTDLNQPVSVYMTPKERLVTVREGEAREVVLAKMHEKRVEKALVVDDEFHLIGMITVKDFQKAEAKPNACKDEQGRLRVGAAVGAGAGNEERVDALVAAGVDVLLIDSSHGHSEGVLQRIRETRAKYPDLQIIGGNVATAAGARALAEAGCSAVKVGIGPGSICTTRIVTGVGVPQITAVADAVEALEGTGIPVIADGGIRFSGDIAKAIAAGASAVMVGSMLAGTEESPGEIELYQGRSYKSYRGMGSLGAMSKGSSDRYFQSDNAADKLVPEGIEGRVAYKGRLKEIIHQQMGGLRSCMGLTGCGTIDELRTKAEFVRISGAGIQESHVHDVTITKESPNYRLGS

SEQ ID NO: 3 - DNA, 1467 bp, Escherichia coli
atgctacgtatcgctaaagaagctctgacgtttgacgacgttctcctcgttcctgctcactctaccgttctgccgaatactgctgacctcagcacccagctgacgaaaactattcgtctgaatatccctatgctttccgcagcaatggataccgtaacggaagcgcgcctggctattgctctggctcaggaaggcggtatcggctttatccacaaaaacatgtccattgaacgccaggcagaagaagttcgccgtgtgaaaaaacacgaatctggtgtggtgactgatccgcagactgttctgccaaccacgacgctgcgcgaagtgaaagaactgaccgagcgtaacggttttgcgggctatccggtcgttaccgaagaaaacgaactggtgggtattatcaccggtcgtgacgtgcgttttgttaccgacctgaaccagccggttagcgtttacatgacgccgaaagagcgtctggtcaccgtgcgtgaaggtgaagcccgtgaagtggtgctggcaaaaatgcacgaaaaacgcgttgaaaaagcgctggtggttgatgacgaattccacctgatcggcatgatcaccgtgaaagacttccagaaagcggaacgtaaaccgaacgcctgtaaagacgagcaaggccgtctgcgtgttggtgcagcggttggcgcaggtgcgggtaacgaagagcgtgttgacgcgctggttgccgcaggcgttgacgttctgctgatcgactcctcccacggtcactcagaaggtgtactgcaacgtatccgtgaaacccgtgctaaatatccggatctgcaaattatcggcggcaacgtggcaacagctgcaggtgcacgcgctctggcagaagctggttgcagtgcggttaaagtcggcattggccctggctctatctgtacaactcgtatcgtgactggcgtcggtgttccgcagattaccgctgttgctgacgcagtagaagccctggaaggcaccggtattccggttatcgctgatggcggtattcgcttctccggcgacatcgccaaagctatcgccgctggcgcaagcgcggtgatggtaggttccatgctggcgggtactgaagaatctccgggtgaaatcgaactctaccagggccgttcttacaaatcttaccgtggtatgggttccctgggcgcgatgtccaaaggttcctctgaccgttatttccagagcgataacgctgccgacaaactggtgccggaaggtatcgaaggtcgcgtagcctataaaggtcgcctgaaagagatcattcaccagcagatgggcggcctgcgctcctgtatgggtctgaccggctgtggtactatcgacgaactgcgtactaaagcggagtttgtacgtatcagcggtgcgggcattcaggaaagccacgttcacgacgtgaccattactaaagagtccccgaactaccgtctgggctcctga